(19) United States
(12) Patent Application Publication
(10) Pub. No.: US 2011/0313229 A1
(43) Pub. Date: Dec. 22, 2011

(54) Therapy Targeting Cancer Stem Cells

(76) Inventors: Kiminobu Sugaya, Winter Park, FL (US); Angel Alvarez, Chicago, IL (US); Sergey Bushnev, Altamonte Springs, FL (US); Nicholas G. Avgeropoulos, Orlando, FL (US)

(21) Appl. No.: 13/055,542

(22) PCT Filed: Jul. 24, 2009
(86) PCT No.: PCT/US2009/051646

§ 371 Date: Sep. 8, 2011

Related U.S. Application Data
(60) Provisional application No. 61/083,273, filed on Jul. 24, 2008.

(51) Int. Cl. A61N 5/00; C12N 5/095; A61P 37/04; A61K 31/713; A61P 35/00; A61K 39/00; C12N 5/0784

(52) U.S. Cl. 600/1; 424/277.1; 435/374; 435/375; 514/44 A

(57) ABSTRACT

Disclosed herein are new immunotherapy methods that involve the isolation of cancer stem cells from tumor tissue and use of the cells either directly or indirectly through proteins or other factors associated with the cells to activate antigen presenting cells. The activated antigen presenting cells are useful as a therapy against the tumor. Also disclosed herein are novel methods of isolating and characterizing cancer stem cells and producing individual cancer stem cell lines. Dendritic cell lines are also disclosed herein.

### RELATED APPLICATIONS

This application claims priority to U. S. Ser. No. 61/083,273 filed Jul. 24, 2008, which is incorporated herein in its entirety.

### BACKGROUND

Treatment for glioblastoma, the most common adult glioma, has expanded, but has not significantly improved the prognosis of patients with this aggressive form of cancer. These tumors contain a heterogenous population of cells, including a subpopulation of cancer stem cells. Unlike normal adult stem cells that are important in cellular repair and homeostasis, cancer stem cells fail to develop properly. Cancer stem cells have been shown to promote angiogenesis, ¹ are resistant to radiation² and chemotherapy, ³ and have the ability to reform tumors. ^{4, 5}.

Glioblastoma multiforme tumors are aggressive gliomas that demonstrate strong resistance to currently available chemotherapy options and frequent reoccurrence following surgery. Following diagnosis, median survival times have been reported between 20 and 36 weeks with surgery alone or combined with radiation, respectively for GBM patients.⁶⁻¹⁰ Median survival times may be increased to up to nearly 15 months if over 98% of the tumor is removed¹¹ or chemotherapy is integrated with surgery and radiation.^{12, 13} Unfortunately, there has been little improvement in survival relative to the original documented average span of 44-52 weeks over 80 years ago.¹⁴

The heterogeneity of these tumors, and particularly the existence of a subpopulation of cancer stem cells, are believed to be critical to the tumorgenic process.^{4, 15, 16} Earlier studies have demonstrated the existence of a subpopulation of cancer stem cells, identified as being positive for the surface marker CD133, within glioblastoma tumors that are able to give rise to new tumors following transplantation into nude mice.^{4, 15-18} Interestingly, transplantation of cancer cells that are negative for CD133 did not appear to form tumors upon transplantation.¹⁸ These CD133 positive cancer stem cells and have been compared to human neural stem cells both on growth properties and gene expression.^{15, 16, 18} However, many of these comparative studies have been carried out using fetal neural stem cells rather than endogenous adult neural stem cells.⁵ All studies that cite CD133 to be an adult neural stem cell marker reference research on fetal or embryonic stem cell-derived neural stem cells.¹⁹⁻²² This distinction may be important because non-fetal adult neural stem cells, at least in the subventricular zone, do not express CD 133 and have not been as well characterized.²³ Therefore, previous comparative studies fail to provide valuable information as to the similarity of cancer stem cells to adult neural stem cells. This may be clinically relevant because glioblastomas contain both cancer and cancer stem cells in addition to normal adult neural stem cells that migrate to the tumor.²⁴⁻²⁶ This migratory phenomenon, that is also observed in brain injury,²⁷ has been proposed as a means of anti-cancer gene delivery.^{28, 29}However, if stem cells are to be a viable vehicle for tumor therapies, then more detailed identification is needed to prevent the accidental implantation of cancer stem cells. Moreover, the biology of both cancer and normal stem cells may be important in understanding cognitive impairments observed in many brain tumor patients and potential cognitive side effects from therapy. ^{17, 30} The ability for cancer stem cells to undergo tumor genesis, combined with the resistance these cells have for radiation and chemotherapy,^{2, 3, 31} is of particular clinical importance given the propensity of gliomas to reemerge following surgery and therapy. The invention embodiments described facilitate better distinction between normal stem cells and cancer stem cells as well as a means of isolating them from patient tumors for potential autologous therapies.

Treatment of gliomas, for example, is particularly challenging given the presence of the blood brain barrier that limits drug deliver.^{32, 33} Cell-based therapies are an attractive option since they may be able to migrate to the tumor and induce cell death, but selecting a target within a heterogenous population may have limited success. Immunotherapy, that exploits the immune systems ability to target foreign cells, involving dendritic cells activated against gliomas has been a proposed therapeutic option in treating GBM^{34, 35} and small scale studies suggest some benefit.³⁶⁻³⁹ However, it is uncertain if directing immunotherapy against these tumor stem cells will be effective, since previous work has demonstrated the ability of these glioma cells to evade immune attack.^{40, 43} Several reports suggest that these tumors have intrinsic immunosuppressive properties,^{40, 44-46} implicating the role of tumor gangliosides.^{42, 43} Dendritic cells, for example, show impaired maturation in the presence of glioma cells and in patients with glioblastoma.^{47, 48} What has not been examined is the influence of sub-populations of tumor cells on immune suppression. If cancer stem cells display immune resistance, then they may evade conventional cell-based therapies. Earlier work has suggested that the cells may avoid immune detection by not expressing MHC-I or NK ligands⁴¹ while another study showed cancer stem cell-like cells displayed higher levels of MHC compared to adherent tumor cells and were used to activate dendritic cells that lowered tumor load.⁴⁹ Additionally, it is unknown if immunotherapy against cancer stem cells will have the unintended effect of destroying normal adult stem cells, potentially leading to greater cognitive impairments. Successful immune-based therapies will have to selectively target cancer stem cells, while avoiding normal stem cells. It is uncertain if this will be possible and conflicting reports on distinguishing both cell populations adds to the confusion. For example, studies examining MHC expression in normal neural stem cells also has yielded mixed results. Odeberg (2005)⁵⁰ reports low immunogenicity despite high MHC-I and MHC-II expression, while a recent study suggests neural stem cells have rather low MHC expression but are nonetheless able to activate peripheral lymphocytes.⁵¹ It is likely that immunogenicity may be more complicated with potential changes in MHC depending on proliferative state⁵² and possible immune modulation through secreated growth factors like transforming growth factor-beta.⁵¹

### GENERAL DESCRIPTION

In certain embodiments, this invention is directed to labeling, isolating and expanding subpopulations of cells within a tumor sample. Accomplishing this makes possible the study of the affects of anti-tumor compounds on each subpopulation, but also enables the use of isolated cells for genetic engineering for anti-tumor therapies.

In certain embodiments, in vivo examination of anti-cancer activity of dendritic cells in a mouse model of glioblastoma serves as a foundation for clinical immunotherapy Immunotherapy targeting cancer stem cells is a novel approach in treating tumors containing these highly aggressive and chemoresistant cells. Furthermore, the inventors have developed protocols that are used to demonstrate that patient-derived stem cells or stem cell-like cells derived from a cell line can generate tumors in rodents following transplantation.^{4, 15, 18, 49}

### Isolation

Isolation of cells may be accomplished by positive selection, negative selection or through histological/growth characteristics. Known markers or discovered markers specific to cancer stem cells are implemented to isolate cancer stem cells from other tumor cell types. In one embodiment, markers such CD133 or CD 45, or other markers may be used for positive selection of cells, such as through flow cytometry or magnetic separation. Conversely, markers absent in cancer stem cells, but present in other cells in tumor, may be used to negatively select out cells other than cancer stem cells.

In a specific embodiment, markers to cancer stem cells are identified. Initially, antibodies are tested to determine if normal neural stem cells and tumor cells lines express these proteins. Antibodies against individual surface markers are purchased, and incubated with preserved cells grown in a cell culture incubator. Immunohistochemical staining of both in vitro human neural stem cell and tumor cells lines using the selected antibodies determines if a particular protein can represent a novel target. A successful candidate is a protein that is highly expressed in one population of cells but not another (eg. Highly expressed in normal human neural stem cells but not in tumor cell lines or vice versa). Paraffin-embedded primary tumor samples will also be used to demonstrate expression of novel protein targets within the tumor. Once at least one successful antibody per group is determined, those antibodies are used to select out subpopulations of cells from tumor samples. This may be accomplished by attaching magnetic particles to antibodies and incubating the conjugated antibodies with cells isolated from the tumor. Following incubation, the cells are run through a magnetic column to separate out cells attached to a magnetic antibody (because of expression of a target surface protein) and non-attached cells will flow through the column. This technique enables purification of individual cell populations within the tumor for further study.

Furthermore, different cells in a tumor sample may be isolated based on their histological or growth characteristics. For example, cells from a tumor sample may be adherent to surfaces compared to other cells. Adherent cells are in most cases more differentiated tumor cells not cancer stem cells. Cancer stem cells also may have a propensity to form spheres. Cells tending to form spheres can be selected apart from cells not tending to form spheres. Cells may also be isolated based on the hanging-drop method. Tissue Engineering, Second Edition, Hauser and Fussenegger, 2007, Human Press.

### Testing of Isolated Cells

The identification and isolation of cancer stem cells enable the determination of agents that are particularly active against of a cancerous condition of a patient in need. According to another embodiment, the invention is directed to a method of identifying optimal chemotherapeutic agents (and/or radiation treatments) for treating a target cancer. The method includes isolating cancer stem cells from a patient and subjecting the cancer stem cells to one or more chemotherapeutic agents. Those agents having an adverse effect, or a conversely, a proliferating effect (or stimulating effect, which will be discussed below in connection with a co-therapies), on the cancer stem cells, are determined to be select agents for treating the cancerous condition. An adverse effect includes inhibition of growth or division cells and/or killing effect on the cells. The chemotherapeutic agents may be known or later developed. Agents to be tested include but are not limited to, the chemotherapeutic agents discussed below.

Categories of Chemotherapeutic Agents

Most chemotherapy agents and medications work by interfering with DNA synthesis or function. Each chemotherapy drug works during different phases of the cell cycle. Based on their action, chemotherapy agents can be classified as cell-cycle specific agents (effective during certain phases of cell cycle) and cell-cycle nonspecific agents (effective during all phases of cell cycle). Depending on their characteristics and nature of treatment, chemotherapy agents can be categorized as alkylating agents, antimetabolites, anthracyclines, antitumor antibiotics, monoclonal antibodies, platinums, or plant alkaloids. Here, we discuss the main features of each of these categories.

Alkylating Agents

Alkylating agents are one of the earliest and most commonly used chemotherapy agents used for cancer treatments. Their use in cancer treatments started in early 1940s. Majority of alkaline agents are active or dormant nitrogen mustards, which are poisonous compound initially used for certain military purposes. Chlorambucil, Cyclophosphamide, CCNU, Melphalan, Procarbazine, Thiotepa, BCNU, and Busulfan are some of the commonly used alkylating agents.

The following three groups are almost always considered “classical”.

Nitrogen mustards
Cyclophosphamide
Mechlorethamine or mustine (HN2)
Uramustine or uracil mustard
Melphalan
Chlorambucil
Ifosfamide

Nitrosoureas
Carmustine
Streptozocin

Alkyl sulfonates
Busulfan

Thiotepa and its analogues are usually considered classical, but can be considered nonclassical.

Although they might differ in their clinical activity, action mechanism of all alkylating agents is the same. These agents work directly on the DNA and prevent the cell division process by cross-linking and breaking the DNA strands and causing abnormal base pairing. When a DNA is altered in this manner, undesired cellular activity comes to a halt and the cell dies eventually.

Alkylating chemotherapy drugs are effective during all phases of cell cycle. Therefore, they are used to treat a large number of cancers. However, they are more effective in treating slow-growing cancers such as solid tumors and leukemia.

Long term use of alkylating agents can lead to permanent infertility by decreasing sperm production in males, and causing menstruation cessation in females. Many alkylating agents can also lead to secondary cancers such as Acute Myeloid Leukemia, years after the therapy.

Nonclassical

Certain alkylating agents are sometimes described as “nonclassical”. There is not a perfect consensus on which items are included in this category, but generally they include:

procarbazine

altretamine

Antimetabolites

Structure of antimetabolites (antineoplastic agents) is similar to certain compounds such as vitamins, amino acids, and precursors of DNA or RNA, found naturally in human body. Antimetabolites help in treatment cancer by inhibiting cell division thereby hindering the growth of tumor cells. These agents get incorporated in the DNA or RNA to interfere with the process of division of cancer cells.

Antimetabolites were first discovered in the year 1948, when Dr. Sidney Farber found that folic acid analog can reduce childhood leukemia. Out of 16 patients he tested, 10 displayed hematologic improvement. This discovery laid the foundation that enabled scientist to synthesize many new agents that could inhibit biological enzymatic reactions.

Antimetabolites are found to be useful in treating chronic and acute cases of leukemia and various tumors. They are commonly used to treat gastrointestinal tract, breast, and ovary tumors.

Methotraxate, which is a commonly used antimetabolites chemotherapy agent, is effective in the S-phase of the cell cycle. It works by inhibiting an enzyme that is essential for DNA synthesis.

6-mercaptopurine and 5-fluorouracil (5FU) are two other commonly used antimetabolites. 5-Fluorouracil (5-FU) works by interfering with the DNA components, nucleotide, to stop DNA synthesis. This drug is used to treat many different types of cancers including breast, esophageal, head, neck, and gastric cancers. 6-mercaptopurine is an analogue of hypoxanthine and is commonly used to treat Acute Lymphoblastic Leukemia (ALL).

Other popular antimetabolite chemotherapy drugs are Thioguanine, Cytarabine, Cladribine. Gemcitabine, and Fludarabine.

Azacitidine

Azathioprine

Capecitabine

Cytarabine

Doxifluridine

Fluorouracil

Gemcitabine

Mercaptopurine

Methotrexate

Tioguanine (formerly Thioguanine)

Anthracyclines

Anthracyclines were developed between 1970s and 1990s and are daunosamine and tetra-hydronaphthacenedione-based chemotherapy agents. These compounds are cell-cycle nonspecific and are used to treat a large number of cancers including lymphomas, leukemia, and uterine, ovarian, lung and breast cancers.

Anthracyclines drugs are developed from natural resources. For instance, daunorubicin is developed by isolating it from soil-dwelling fungus Streptomyces. Similarly, Doxorubicin, which is another commonly used anthracycline chemotherapy agent, is isolated from mutated strain of Streptomyces. Although both the drugs have similar clinical action mechanisms, doxorubicin is more effective in treating solid tumors. Idarubicin, Epirubicin, and Mitoxantrone are few of the other commonly used anthracycline chemotherapy drugs.

Anthracyclines work by forming free oxygen radicals that breaks DNA strands thereby inhibiting DNA synthesis and function. These chemotherapeutic agents form a complex with DNA and enzyme to inhibit the topoisomerase enzyme. Topoisomerase is an enzyme class that causes the supercoiling of DNA, allowing DNA repair, transcription, and replication.

One of the main side effects of anthracyclines is that it can damage cells of heart muscle along with the DNA of cancer cell leading to cardiac toxicity.

Available agents include:

Daunorubicin (Daunomycin)

Daunorubicin (liposomal)

Doxorubicin (Adriamycin)

Doxorubicin (liposomal)

Epirubicin

Idarubicin

Valrubicin, used only to treat bladder cancer

Since they are antibiotics, anthracyclines can kill or inhibit the growth of bacteria, but because they are so toxic to humans, they are never used to treat infections.

Antitumor Antibiotics

Antitumor antibiotics are also developed from the soil fungus Streptomyces. These drugs are widely used to treat and suppress development of tumors in the body. Similar to anthracyclines, antitumor antibiotics drugs also form free oxygen radicals that result in DNA strand breaks, killing the growth of cancer cells. In most of the cases, these drugs are used in combination with other chemotherapy agents.

Bleomycin is one of the commonly used antitumor antibiotic used to treat testicular cancer and hodgkin's lymphoma.

The most serious side effect of this drug is lung toxicity that occurs when the oxygen radical formed by the antitumor antibiotics damages lung cells along with the cancer cells.

Monoclonal Antibodies

Monoclonal antibodies are one of the newer chemotherapy agents approved for cancer treatment by the Food and Drug Administration (FDA) in 1997. Alemtuzumab (Campath), Bevacizumab (Avastin), Cetuximab (Erbitux), Gemtuzumab (Mylotarg), Ibritumomab (Zevalin), Panitumumab (Vectibix), Rituximab (Rituxan), Tositumomab (Bexxar), and Trastuzumab (Herceptin) are some of the FDA approved monoclonal drugs used in chemotherapeutic cancer treatments.

The treatment is known to be useful in treating colon, lung, head, neck, and breast cancers. Some of the monoclonal drugs are used to treat chronic lymphocytic leukemia, acute myelogenous leukemia, and non-Hodgkin's lymphoma.

Monoclonal antibodies work by attaching to certain parts of the tumor-specific antigens and make them easily recognizable by the host's immune system. They also prevent growth of cancer cells by blocking the cell receptors to which chemicals called ‘growth factors’ attach promoting cell growth.

Monoclonal antibodies can be combined with radioactive particles and other powerful anticancer drugs to deliver them directly to cancer cells. Using this method, long term radioactive treatment and anticancer drugs can be given to patients without causing any serious harm to other healthy cells of the body.

Platinums

Platinum-based natural metal derivatives were found to be useful for cancer treatments around 150 years ago with the synthesis of cisplatin. However, there clinical use did not commence until 30 years ago. Platinum-based chemotherapy agents work by cross-linking subunits of DNA. These agents act during any part of cell cycle and help in treating cancer by impairing DNA synthesis, transcription, and function.

Cisplatin, although found to be useful in treating testicular and lung cancer, is highly toxic and can severely damage the kidneys of the patient. Second generation platinum-complex carboplatin is found to be much less toxic in comparison to cisplatin and has fewer kidney-related side effects. Oxaliplatin, which is third generation platinum-based complex, is found to be helpful in treating colon cancer. Although, oxaliplatin does not cause any toxicity in kidney it can lead to severe neuropathies.

Alkylating-like

Platinum-based chemotherapeutic drugs (termed platinum analogues) act in a similar manner. These agents don't have an alkyl group, but nevertheless damage DNA.They permanently coordinate to DNA to interfere with DNA repair, so they are sometimes described as “alkylating-like”.

Platinum^{[5]}
Cisplatin
Carboplatin
Nedaplatin
Oxaliplatin
Satraplatin
Triplatin tetranitrate
   These agents also bind at N7 of guanine.

Plant Alkaloids

Plant alkaloid chemotherapy agents, as the name suggests, are plant derivatives. They are cell-specific chemotherapy agents. However, the cycle affected is based on the drug used for the treatment. They are primarily categorized into four groups: topoisomerase inhibitors, vinca alkaloids, taxanes, and epipodophyllotoxins. Plant alkaloids are cell-cycle specific, but the cycle affected varies from drug to drug. Vincristine (Oncovin) is a plant alkaloid of interest in mesothelioma treatment.

Topoisomerase Inhibitors

Topoisomerase inhibitors are chemotherapy agents are categorized into Type I and Type II Topoisomerases inhibitors and they work by interfering with DNA transcription, replication, and function to prevent DNA supercoiling.
Type I Topoisomerase inhibitors: These chemotherapy agents are extracted from the bark and wood of the Chinese tree Camptotheca accuminata. They work by forming a complex with topoisomerase DNA. This in turn suppresses the function of topoisomerase.
Camptothecins which includes irinotecan and topotecan are commonly used type I topoisomerase inhibitors, first discovered in the late 1950s.
Type II Topoisomerase inhibitors: These are extracted from the alkaloids found in the roots of May Apple plants. They work in the in the work in the late S and G2 phases of the cell cycle.
Amsacrine, etoposide, etoposide phosphate, and teniposide are some of the examples of type II topoisomerase inhibitors.

Vinca Alkaloids

Vinca alkaloids are derived from the periwinkle plant, Vinca rosea (Catharanthus roseus) and are known to be used by the natives of Madagascar to treat diabetes.

Although not useful in controlling diabetes, vinca alkaloids, are useful in treating leukemias. They are effective in the M phase of the cell cycle and work by inhibiting tubulin assembly in microtubules.

Vincristine, Vinblastine, Vinorelbine, and Vindesine are some of the popularly used vinca alkaloid chemotherapy agents used today. Major side effect of vinca alkaloids is that they can cause neurotoxicity in patients.

Taxanes

Taxanes are plant alkaloids that were first developed in 1963 by isolating it from first isolated from the bark of the Pacific yew tree, Taxus brevifolia in 1963. Paclitaxel, which is the active components of taxanes was first discovered in 1971 and was made available for clinical use in the year 1993.

Taxanes also work in the M-phase of the cell cycle and inhibit the function of microtubules by binding with them. Paclitaxel and docetaxel are commonly used taxanes. Taxanes chemotherapy agents are used to treat a large array of cancers including breast, ovarian, lung, head and neck, gastric, esophageal, prostrate and gastric cancers. The main side effect of taxanes is that they lower the blood counts in patients. Spindle inhibitors.

Epipodophyllotoxins

Epipodophyllotoxins chemotherapy agents are extracted from the American May Apple tree (Podophyllum peltatum). Recently, it has been found in more quantities in the endangered Himalayan May Apple tree.

Etoposide and Teniposide are commonly used epipodophyllotoxins chemotherapy agents which are effective in the G1 and S phases of the cell cycle. They prevent DNA replication by stopping the cell from entering the G1 phase and stop DNA replication in the S phase.

### Immunotherapy

According to another embodiment, the invention pertains to a method of conducting immunotherapy involving the administration of activated antigen presenting cells. In another embodiment, the invention involves the creation of antigen presenting cells (APCs) activated against cancer stem cells. As used herein, antigen presenting cells include but are not limited to dendritic cells, macrophages or natural killer cells. Other examples of cells that could serve as antigen presenting cells, include fibroblasts, glial cells and microglial cells.

In one example, dendritic cells are activated against markers and antigens present in cancer stem cells. APCs are contacted with the marker or antigen, they are taken into the cell, processed and then presented on the surface of the cell. In another example, mRNA or DNA in CSCs is subjected to APCs, which also results in an activation against the CSCs from which the mRNA and/or DNA was procured. In another example, dendritic cells are activated by fusion with a CSC. The antigen presenting cells take in and digest the cancer stem cells by phagocytosis and/or endocytosis. Alternatively, or in conjunction with phagocytosis and/or endocytosis, the dendritic cells are subjected to electrical current in the presence of the CSCs.

In another embodiment, a tumor sample containing multiple cell types is procured from a subject. As has been discussed herein, it is the inventors' belief that if cancer stem cells can be preferentially targeted over other cells in a tumor this will dramatically improve cancer therapy. Accordingly, cancer stem cells are isolated or enriched from the tumor sample. Tumor samples may be procured from an allogeneic source, i.e., a subject of the same species but other than the subject into which activated antigen presenting cells are administered. In other embodiments, the tumor samples are procured from an autologous source. For example, tumor cells are removed from a cancer subject, the cells are used to activate antigen presenting cells ex vivo and then the activated cells are administered to the cancer subject.

### Combination Therapy

The inventors have realized that cancer stem cells are somewhat inactive which make them difficult to treat with many chemotherapeutic agents. Not to be bound by any theory, it is postulated that cancer stem cells are likely the source of cells that leads a relapse of a cancerous condition after a patient has been in “remission”. The inventors have realized that if these cells could be stimulated to become active, it would make them more vulnerable to chemotherapy and/or radiation treatments.

Accordingly, another embodiment of the invention pertains to treating a patient experiencing a cancer condition with a stem cell stimulating agent. Simultaneously, or sequentially, the patient is treated with a known chemotherapy agent and/or radiation treatment. The stem cell stimulating agent may include, but are not limited to, compounds such as those described in U.S. patent application Ser. No. 11/563,891. Other stimulating agents include those found in U.S. patent application Ser. No. 11/968,393. Also, it has been found that cancer stem cells express nanog which may keep them in an undifferentiated state. Thus, in an alternative embodiment, cancer stem cells are treated with an agent that blocks or inhibits nanog. For example, the agent may include an siRNA or ribozyme directed to nanog. See U.S. patent application Ser. Nos. 11/258,401 and 11/258,360 for techniques for constructing siRNA against nanog. See

U.S. Pat. No.7,462,602, for example, for information concerning ribozymes.

Cancer Stem Cell Lines

In a further embodiment, the subject invention pertains to a plurality of cancer stem cell lines and a facility for storage of such lines. This embodiment is based on the inventors' realization that there is a need for a convenient systematic access to different cancer stem cell lines. The inventors have realized that the ability to identify cancer stem cell lines derived from various tumor types will be exceedingly useful for identifying specific markers for distinguishing cancer stem cells from other cells in a given cancer type. Different cancer stem cell lines will be useful for testing various compounds for their effect on the growth and/or survival of the specific cancer stem cell type. This in turn, will lead to the discovery of potential new cancer therapies. Subjects from which cancer stem cells are procured for establishing a given cell line may be human or nonhuman vertebrates.

According to another embodiment, cancer stem cells are harvested, catalogued according to predetermined characteristics, e.g., phenotypic information, morphological characteristics, differentiation profile, blood type, major histocompatibility complex, disease state of donor, or genotypic information (e.g. single nucleated polymorphisms, ‘SNPs’ of a specific nucleic acid sequence associated with a gene, or genomic or mitochondrial DNA), and stored under appropriate conditions (typically by freezing) to keep the cancer stem cells alive and functioning. Other characteristics may include, resistance to chemotherapies, production of membrane channels that confer drug resistance, surface markers and surface receptors. Cataloguing may constitute creating a centralized record of the characteristics obtained for each cell population, such as, but not limited to, an assembled written record or a computer database with information inputted therein. Essentially, this embodiment pertains to the production of a stem cell bank. The cancer stem cell bank facilitates the selection from a plurality of samples of a specific stem cell sample suitable for a researcher's needs. Thus, another embodiment of the subject invention pertains to a cancer stem cell bank comprising a plurality of cancer stem cell samples obtained from separate sources and which are characterized and catalogued according to at least one predetermined characteristic. An additional embodiment pertains to a method of establishing a cancer stem cell bank comprising collecting cancer stem cell samples from multiple sources; cataloguing the samples according to at least one predetermined characteristic and storing the cancer stem cells under conditions that keep cells viable.

According to a specific embodiment, the subject invention pertains to a cancer stem cell banking system comprising a plurality of cancer stem cell populations disposed in individual containers under conditions to keep said stem cell populations viable; a database computer comprising at least one processing module, a display, and a storage medium comprising information of at least one characteristic for each of said cancer stem cell populations; and at least one program code module for causing said information to be viewable on said display upon command by a user. In a specific embodiment, the invention pertains to a cancer stem cell banking system wherein the cancer stem cell populations comprise cancer stem cells obtained from subjects who have a cancer condition, whether in the form of a tumor or otherwise. Cancer stem cells are harvested from different subjects having different cancers, and the cancer stem cells are characterized. The characteristic(s) is/are inputed into the database computer. In addition, or alternatively, cancer stem cells are characterized based on a specific phenotype not necessarily associated with a disease condition.

### Activated Dendritic Cell Lines

In another embodiment, cell lines of activated dendritic cells are produced. A population of dendritic cells may be produced that are activated against a particular cancer stem cell sample. Unfortunately, it is often the case that cancer patients have a very short term of life if therapy is not immediately forthcoming In some circumstances, methods of isolating cancer stem cell, isolating antigen presenting cells and activating the antigen presenting cells takes time that patients cannot afford. Accordingly, a dendritic cell bank that provides a storage of cells that can be immediately used for immunotherapy will be of dramatic benefit to certain patients.

Cancer stem cell samples may be obtained from different cancer/tumor types. Moreover, similar to the cancer stem cell lines, activated dendritic cell lines may be catalogued according to predetermined characteristics, e.g., phenotypic information, morphological characteristics, differentiation profile, blood type, major histocompatibility complex, disease state of donor or cancer type, or genotypic information (e.g. single nucleated polymorphisms, ‘SNPs’ of a specific nucleic acid sequence associated with a gene, or genomic or mitochondrial DNA), and stored under appropriate conditions (typically by freezing) to keep the activated dendritic cells alive and functioning. In one embodiment, the activated dendritic cell lines are catalogued according to the cancer type pertaining to the source of the cancer stem cells used to activate the dendritic cells. Examples of cancer types include but are not limited to:

Cancer Type
Bladder Cancer
Breast Cancer
Colon and Rectal
Endometrial Cancer
Kidney Cancer
Leukemia
Lung Cancer
Melanoma
Non-Hodgkin's Lymphoma
Pancreatic Cancer
Prostate Cancer
Skin Cancer (non-melanoma)
neuroblastoma
astrocytoma
CNS lymphoma
Glioblastoma multiforme
craniopharyngioma
Metastic brain tumors
meningioma
Pituitary tumors
Thyroid Cancer

In a more specific embodiment, the activated dendritic cell lines are catalogued based on the cancer/tumor type used for activation along with at least one other characteristic, such as phenotypic information, morphological characteristics, differentiation profile, blood type, major histocompatibility complex, or genotypic information (e.g. single nucleated polymorphisms, ‘SNPs’ of a specific nucleic acid sequence associated with a gene, or genomic or mitochondrial DNA

### EXAMPLES

### Example 1

### Cell Culture and Isolation

Human glioblastoma cells are removed from patients undergoing treatment surgery, who have provided informed consent for the study. Brain tumors are measured and graded according to WHO criteria⁷¹⁻⁷³ by a trained pathologist and excess tissue used for experimentation. Surgically removed tumor specimens are washed, minced, and enzymatically dissociated, then plated at densities of 2×10⁶ live cells inside a 75 cm² flask containing resuspension medium of DMEM/F12 supplemented with 10% fetal bovine serum within an hour of surgery. Following an initial expansion in a monolayer, the tumor cells are switched to a defined serum-free NSC Basal medium supplemented with 20 ng/ml of basic fibroblast growth factor (FGF-2) and 20 ng/ml of epidermal growth factor (EGF) to generate neural sphere formation. This culturing system will generate cells with two distinct growth properties, adherent cells and floating sphere-forming cells. Adherent cells are likely differentiated tumor cells with limited proliferative potential. Floating neural spheres contain multipotent stem cells. Following sphere formation, colonies are dissociated and individual cells are isolated and placed in separate wells of a 96-well plate containing NSC medium to examine the ability to generate clonal neural spheres. Cell isolation is performed using separation technique with magnetic-bead fluorescent-label conjugated antibodies to positively select out a specific surface protein, like CD133 using magnetic separation or flow cytometry. In addition to the sphere-forming assay, cells are analyzed using quantitative real-time PCR for expression of neural stem cell genes, stem cell transcription factors, tumor cell markers, and genes associated with neural and glial differentiation. Additional characterization is performed using known stem cell surface markers MCM2 and 2F7 to determine if they are differentially expressed between cancer and normal neural stem cells. Ganglioside expression is assessed to determine if cancer cells and cancer stem cells express known immune-suppressive gangliosides or if they hide from the immune system by expressing glycoconjugates observed in normal adult neural stem cells.

### Example 2

### RNA Isolation and Quantitative Real-Time PCR

cell culture medium is removed from cells and RNA extraction is performed using a commercially available TRIZOL reagent. RNA concentration is measured using spectrophotometry. Gene expression is measured by quantitative real-time PCR (qRT-PCR) using gene specific primers. Primers are tested for amplification efficiency using standardization curves and expression levels are determined relative to both control samples and internal housekeeping genes.

### Example 3

### Dendritic Cell Differentiation and Vaccination

Dendritic cells are derived from peripheral blood samples or human umbilical cord blood, depending on availability and differentiated into dendritic cells using previously established protocols.^{49, 60} Initially, cells are isolated and cultured in 25 cm² cell culture flasks with RPMI 1640 supplemented with albumin for 2 hours to allow cell adhesion. Following a 2-hour incubation at 37° C., non-adherent cells are removed and the medium replaced to facilitate immature dendritic cell differentiation. Cell culture medium consisting of serum-free and defined X-VIVO 15 medium supplemented with 100 ng/ml of GM-CSF, 25 ng/ml of IL-4, and 2% human albumin, all commercially available, is used for human immature dendritic cell development for 7 days. X-VIVO 15 medium is selected because it has already been certified as clinical grade.⁶⁰

### Example 4

### Functionality of Dendritic Cells

Immature and mature dendritic cells are cultured in the presence of lysine-fixable, FITC-conjugated dextran to examine the function of mannose-receptor mediated endocytosis.⁵⁹⁻⁶⁰ Culturing dendritic cells for up to 30 minutes using 1 mg/ml of conjugated dextran allows cells that take up the molecule to be analyzed using flow cytometry. Following incubation with FITC-dextran, cells are washed with a solution of 1% FCS and 0.02% sodium azide in PBS. Cells are collected following FACS using the cell culture medium the cells were grown in. Phagocytosis of tumor cells is evaluated using time-lapse imaging in an enclosed culture chamber placed under an inverted microscope. Cells are labeled using separate red and green dyes or fluorescent genes delivered to the tumor cells and dendritic cells using lentiviruses, respectively. This will allow the visualization and FACS analysis of dendritic cells that phagocytized labeled tumor cells. ^{60, 63}Mature dendritic cells are tested for markers MHC I and II, CD11 c, CD80, and CD86. Dendritic cells are activated against tumor cells or tumor stem cells by using an electroporation device by placing both cell types into a cuvett and pulsed with an electric charge to produce fused cell hybrids.

### Example 5

### Irradiation of Cancer Stem Cells Prior to Fusion

Prior to fusion with dendritic cells, cancer stem cells are irradiated to help reduce the potential for tumorgenesis upon transplantation. Previous studies have successfully demonstrated the ability of irradiated cancer cells as a safe and effective source for dendritic cell therapy.69, 74, 75 Cells are exposed to 200 Gy of gamma irradiation, a high dose that does not appear to prevent effective cell fusion,⁷⁵ but would be better suited for cells that have shown resistance to irradiation.^{2, 31} Additionally, inducing apoptosis through irradiation elicits a greater therapeutic response when fused to dendritic cells than cancer stem cells that undergo necrosis, as in freeze-thaw cycles.⁶⁹

### Example 6

### Animal Studies

Athymic male nude mice are used for experimentation (n=18, 6 per group). Animals are housed at room temperature in a clean room with filtered cages, containing adequate food and water, and no more than 2 animals per cage. Cancer stem cells (1×10̂5 cells) are injected intracranially in mice to generate gliomas. Subsequently, mice will receive either 1×10̂6 GFP positive dendritic cells activated against tumor stem cells or non-activated dendritic cells as a control at days 7, 14, and 21. Mice are monitored for one month post-immune cell transplantation, at which point, surviving animals in all groups are sacrificed by an overdose of anesthesia (sodium pentobarbital, 70 mg/kg) and perfused with phosphate buffered saline (PBS) followed by 4% paraformaldehyde. Brains are removed and placed into 4% paraformaldehyde fixative containing 20% sucrose overnight then sliced into 30 μm coronal section using a cryo-microtome. The sections are then washed, immunostained with specific antibodies, mounted on glass slides, which are covered with Vectashield with DAPI for observation using the fluorescent microscope. GFP-labeled dendritic cells are visualized using a FITC filter. Ganglioside expression and localization is assessed to determine if there is evidence of ganglioside induced dendritic cell death or correlation with treatment outcomes.

Some brain tissue containing tumor implants is sliced to 4-5 μm, embedded in paraffin for routine histology evaluation. Paraffin sections are stained by H&E and assessed for features of glioblastoma, extent of tumor necrosis, mitotic activity, and density of apoptotic bodies. Based on the preliminary finding, select paraffin blocks are stained by immunohistochemistry to detect neoplastic expression of glial fibrillary acidic protein (GFAP) and MIB-1 (Ki-67) antigen and TUNEL (for apoptosis).

### REFERENCES

1. Bao, S. et al. Stem cell-like glioma cells promote tumor angiogenesis through vascular endothelial growth factor. Cancer Res 66, 7843-8 (2006).

2. Bao, S. et al. Glioma stem cells promote radioresistance by preferential activation of the DNA damage response. Nature 444, 756-60 (2006).

3. Liu, G. et al. Analysis of gene expression and chemoresistance of CD133+ cancer stem cells in glioblastoma. Mol Cancer 5, 67 (2006).

4. Yuan, X. et al. Isolation of cancer stem cells from adult glioblastoma multiforme. Oncogene 23, 9392-400 (2004).

5. Zhang, Q. B. et al. Differentiation profile of brain tumor stem cells: a comparative study with neural stem cells. Cell Res 16, 909-15 (2006).

6. Walker, M. D., Strike, T. A. & Sheline, G. E. An analysis of dose-effect relationship in the radiotherapy of malignant gliomas. Int J Radiat Oncol Biol Phys 5, 1725-31 (1979).

7. Simpson, J. R. et al. Influence of location and extent of surgical resection on survival of patients with glioblastoma multiforme: results of three consecutive Radiation Therapy Oncology Group (RTOG) clinical trials. Int J Radiat Oncol Biol Phys 26, 239-44 (1993).

8. Laws, E. R. et al. Survival following surgery and prognostic factors for recently diagnosed malignant glioma: data from the Glioma Outcomes Project. J Neurosurg 99, 467-73 (2003).

9. Fine, H. A., Dear, K. B., Loeffler, J. S., Black, P. M. & Canellos, G. P. Meta-analysis of radiation therapy with and without adjuvant chemotherapy for malignant gliomas in adults. Cancer 71, 2585-97 (1993).

10. Huncharek, M. & Muscat, J. Treatment of recurrent high grade astrocytoma; results of a systematic review of 1,415 patients. Anticancer Res 18, 1303-11 (1998).

11. Lacroix, M. et al. A multivariate analysis of 416 patients with glioblastoma multiforme: prognosis, extent of resection, and survival. J Neurosurg 95, 190-8 (2001).

12. Stupp, R. et al. Promising survival for patients with newly diagnosed glioblastoma multiforme treated with concomitant radiation plus temozolomide followed by adjuvant temozolomide. J Clin Oncol 20, 1375-82 (2002).

13. Stupp, R. et al. Radiotherapy plus concomitant and adjuvant temozolomide for glioblastoma. N Engl J Med 352, 987-96 (2005).

14. Tait, M. J., Petrik, V., Loosemore, A., Bell, B. A. & Papadopoulos, M. C. Survival of patients with glioblastoma multiforme has not improved between 1993 and 2004: analysis of 625 cases. Br J Neurosurg, 1-5 (2007).

15. Galli, R. et al. Isolation and characterization of tumorigenic, stem-like neural precursors from human glioblastoma. Cancer Res 64, 7011-21 (2004).

16. Singh, S. K. et al. Identification of a cancer stem cell in human brain tumors. Cancer Res 63, 5821-8 (2003).

17. Noble, M. & Dietrich, J. Intersections between neurobiology and oncology: tumor origin, treatment and repair of treatment-associated damage. Trends Neurosci 25, 103-7 (2002).

18. Singh, S. K. et al. Identification of human brain tumour initiating cells. Nature 432, 396-401 (2004).

19. Uchida, N. et al. Direct isolation of human central nervous system stem cells. Proc Natl Acad Sci U S A 97, 14720-5 (2000).

20. Lee, A. et al. Isolation of neural stem cells from the postnatal cerebellum. Nat Neurosci 8, 723-9 (2005).

21. Kania, G. et al. Somatic stem cell marker prominin-1/CD133 is expressed in embryonic stem cell-derived progenitors. Stem Cells 23, 791-804 (2005).

22. Barraud, P., Stott, S., Mollgard, K., Parmar, M. & Bjorklund, A. In vitro characterization of a human neural progenitor cell coexpressing SSEA4 and CD133. J Neurosci Res 85, 250-9 (2007).

23. Pfenninger, C. V. et al. CD133 is not present on neurogenic astrocytes in the adult subventricular zone, but on embryonic neural stem cells, ependymal cells, and glioblastoma cells. Cancer Res 67, 5727-36 (2007).

24. Aboody, K. S. et al. Neural stem cells display extensive tropism for pathology in adult brain: evidence from intracranial gliomas. Proc Natl Acad Sci U S A 97, 12846-51 (2000).

25. Tang, Y. et al. In vivo tracking of neural progenitor cell migration to glioblastomas. Hum Gene Ther 14, 1247-54 (2003).

26. Zhang, Z. et al. In vivo magnetic resonance imaging tracks adult neural progenitor cell targeting of brain tumor. Neuroimage 23, 281-7 (2004).

27. Goings, G. E., Sahni, V. & Szele, F. G. Migration patterns of subventricular zone cells in adult mice change after cerebral cortex injury. Brain Res 996, 213-26 (2004).

28. Kim, S. K. et al. PEX-producing human neural stem cells inhibit tumor growth in a mouse glioma model. Clin Cancer Res 11, 5965-70 (2005).

29. Kim, S. K. et al. Human neural stem cells target experimental intracranial medulloblastoma and deliver a therapeutic gene leading to tumor regression. Clin Cancer Res 12, 5550-6 (2006).

30. Butler, J. M., Rapp, S. R. & Shaw, E. G. Managing the cognitive effects of brain tumor radiation therapy. Curr Treat Options Oncol 7, 517-23 (2006).

31. Rich, J. N. Cancer stem cells in radiation resistance. Cancer Res 67, 8980-4 (2007).

32. Grossman, S. A. & Batara, J. F. Current management of glioblastoma multiforme. Semin Oncol 31, 635-44 (2004).

33. Muldoon, L. L. et al. Chemotherapy delivery issues in central nervous system malignancy: a reality check. J Clin Oncol 25, 2295-305 (2007).

34. Zhang, J. G. et al. Antigenic profiling of glioma cells to generate allogeneic vaccines or dendritic cell-based therapeutics. Clin Cancer Res 13, 566-75 (2007).

35. Steinman, R. M. & Banchereau, J. Taking dendritic cells into medicine. Nature 449, 419-26 (2007).

36. Khan, J. A. & Yaqin, S. Successful immunological treatment of gallbladder cancer in India--case report. J Zhejiang Univ Sci B 7, 719-24 (2006).

37. Khan, J. A. & Yaqin, S. Dendritic cell therapy with improved outcome in glioma multiforme--a case report. J Zhejiang Univ Sci B 7, 114-7 (2006).

38. Liau, L. M. et al. Treatment of a patient by vaccination with autologous dendritic cells pulsed with allogeneic major histocompatibility complex class I-matched tumor peptides. Case Report. Neurosurg Focus 9, e8 (2000).

39. Saikali, S. et al. Expression of nine tumour antigens in a series of human glioblastoma multiforme: interest of EGFRvIII, IL-13Ralpha2, gp100 and TRP-2 for immunotherapy. J Neurooncol 81, 139-48 (2007).

40. Gomez, G. G. & Kruse, C. A. Mechanisms of malignant glioma immune resistance and sources of immunosuppression. Gene Ther Mol Biol 10, 133-146 (2006).

41. Wu, A. et al. Expression of MHC I and NK ligands on human CD133+ glioma cells: possible targets of immunotherapy. J Neurooncol 83, 121-31 (2007).

42. Tourkova, I. L. et al. Restoration by IL-15 of MHC class I antigen-processing machinery in human dendritic cells inhibited by tumor-derived gangliosides. J Immunol 175, 3045-52 (2005).

43. Chahlavi, A. et al. Glioblastomas induce T-lymphocyte death by two distinct pathways involving gangliosides and CD70. Cancer Res 65, 5428-38 (2005).

44. Couldwell, W. T., Dore-Duffy, P., Apuzzo, M. L. & Antel, J. P. Malignant glioma modulation of immune function: relative contribution of different soluble factors. J Neuroimmunol 33, 89-96 (1991).

45. Parajuli, P., Mathupala, S., Mittal, S. & Sloan, A. E. Dendritic cell-based active specific immunotherapy for malignant glioma. Expert Opin Biol Ther 7, 439-48 (2007).

46. Roth, P. et al. Regeneration and tolerance factor: a novel mediator of glioblastoma-associated immunosuppression. Cancer Res 66, 3852-8 (2006).

47. Kikuchi, T., Abe, T. & Ohno, T. Effects of glioma cells on maturation of dendritic cells. J Neurooncol 58, 125-30 (2002).

48. Ogden, A. T. et al. Defective receptor expression and dendritic cell differentiation of monocytes in glioblastomas. Neurosurgery 59, 902-9; discussion 909-10 (2006).

49. Pellegatta, S. et al. Neurospheres enriched in cancer stem-like cells are highly effective in eliciting a dendritic cell-mediated immune response against malignant gliomas. Cancer Res 66, 10247-52 (2006).

50. Odeberg, J., Piao, J. H., Samuelsson, E. B., Falci, S. & Akesson, E. Low immunogenicity of in vitro-expanded human neural cells despite high MHC expression. J Neuroimmunol 161, 1-11 (2005).

51. Ubiali, F. et al. Allorecognition of human neural stem cells by peripheral blood lymphocytes despite low expression of MHC molecules: role of TGF-beta in modulating proliferation. Int Immunol 19, 1063-74 (2007).

52. Modo, M., Mellodew, K. & Rezaie, P. In vitro expression of major histocompatibility class I and class II antigens by conditionally immortalized murine neural stem cells. Neurosci Lett 337, 85-8 (2003).

53. Piao, J. H. et al. Cellular composition of long-term human spinal cord- and forebrain-derived neurosphere cultures. J Neurosci Res 84, 471-82 (2006).

54. Beier, D. et al. CD133(+) and CD133(-) glioblastoma-derived cancer stem cells show differential growth characteristics and molecular profiles. Cancer Res 67, 4010-5 (2007).

55. Maslov, A. Y., Barone, T. A., Plunkett, R. J. & Pruitt, S. C. Neural stem cell detection, characterization, and age-related changes in the subventricular zone of mice. J Neurosci 24, 1726-33 (2004).

56. Maslov, A. Y. et al. Stem/progenitor cell-specific enhanced green fluorescent protein expression driven by the endogenous Mcm2 promoter. Stem Cells 25, 132-8 (2007).

57. Schubert, W. et al. Characterization and distribution of a new cell surface marker of neuronal precursors. Dev Neurosci 22, 154-66 (2000).

58. Klassen, H., Schwartz, M. R., Bailey, A. H. & Young, M. J. Surface markers expressed by multipotent human and mouse neural progenitor cells include tetraspanins and non-protein epitopes. Neurosci Lett 312, 180-2 (2001).

59. Tarte, K. et al. Generation of virtually pure and potentially proliferating dendritic cells from non-CD34 apheresis cells from patients with multiple myeloma. Blood 90, 3482-95 (1997).

60. Tarte, K., Fiol, G., Rossi, J. F. & Klein, B. Extensive characterization of dendritic cells generated in serum-free conditions: regulation of soluble antigen uptake, apoptotic tumor cell phagocytosis, chemotaxis and T cell activation during maturation in vitro. Leukemia 14, 2182-92 (2000).

61. Shortman, K. & Caux, C. Dendritic cell development: multiple pathways to nature's adjuvants. Stem Cells 15, 409-19 (1997).

62. Banchereau, J. & Steinman, R. M. Dendritic cells and the control of immunity. Nature 392, 245-52 (1998).

63. Albert, M. L. et al Immature dendritic cells phagocytose apoptotic cells via alphavbeta5 and CD36, and cross-present antigens to cytotoxic T lymphocytes. J Exp Med 188, 1359-68 (1998).

64. Scott-Taylor, T. H. et al. Human tumour and dendritic cell hybrids generated by electrofusion: potential for cancer vaccines. Biochim Biophys Acta 1500, 265-79 (2000).

65. Gottfried, E. et al. Characterization of cells prepared by dendritic cell-tumor cell fusion. Cancer Immun 2, 15 (2002).

66. Shimizu, K. et al. Comparative analysis of antigen loading strategies of dendritic cells for tumor immunotherapy J Immunother (1997) 27, 265-72 (2004).

67. Weise, J. B. et al. A dendritic cell based hybrid cell vaccine generated by electrofusion for immunotherapy strategies in HNSCC. Auris Nasus Larynx 31, 149-53 (2004).

68. Weise, J. B. et al. Electrofusion generates diverse DC-tumour cell hybrids for cancer immunotherapy. Anticancer Res 24, 929-34 (2004).

69. Yasuda, T. et al. Dendritic cell-tumor cell hybrids enhance the induction of cytotoxic T lymphocytes against murine colon cancer: a comparative analysis of antigen loading methods for the vaccination of immunotherapeutic dendritic cells. Oncol Rep 16, 1317-24 (2006).

70. Wang, Z. et al. Effect of dendritic cell vaccine against a tongue squamous cell cancer cell line (Tca8113) in vivo and in vitro. Int J Oral Maxillofac Surg 35, 544-50 (2006).

71. Kleihues, P., Soylemezoglu, F., Schauble, B., Scheithauer, B. W. & Burger, P. C. Histopathology, classification, and grading of gliomas. Glia 15, 211-21 (1995).

72. Kleihues, P. et al. The WHO classification of tumors of the nervous system. J Neuropathol Exp Neurol 61, 215-25; discussion 226-9 (2002).

73. Louis, D. N. et al. The 2007 WHO classification of tumours of the central nervous system. Acta Neuropathol (Berl) 114, 97-109 (2007).

74. Marten, A. et al. Allogeneic dendritic cells fused with tumor cells: preclinical results and outcome of a clinical phase I/II trial in patients with metastatic renal cell carcinoma. Hum Gene Ther 14, 483-94 (2003).

75. Trevor, K. T. et al. Generation of dendritic cell-tumor cell hybrids by electrofusion for clinical vaccine application. Cancer Immunol Immunother 53, 705-14 (2004).

U.S. Patent Publications 20070071731; 20060188489; and 20060134789 are cited for further discussion of stem cells, and experimental protocols related thereto.

In reviewing the detailed disclosure which follows, and the specification more generally, it should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

1. A cancer therapy method comprising:
obtaining a sample of cancer stem cells;
activating a sample of antigen presenting cells by subjecting said sample of antigen presenting cells to said sample of cancer stem cells to produce activated antigen presenting cells; and
administering said activated antigen presenting cells to subject in need thereof.

2. The method of claim 1, wherein said sample of antigen presenting cells comprises dendritic cells.

3. The method of claim 1, wherein said sample of cancer stem cells is autologous to said subject.

4. (canceled)

5. (canceled)

6. A method of producing multiple cancer stem cell populations for storage in a cancer stem cell bank comprising:
obtaining cancer stem cells from individual tumor samples from a plurality of subjects to produce a plurality of separate cancer stem cell populations;
cataloguing said cancer stem cell populations according to at least one predetermined characteristic; and
storing said cancer stem cell populations under conditions to maintain viability for more than a week.

7. The method of claim 6, wherein said predetermined characteristic comprises genotypic and/or phenotypic information.

8. The method of claim 6, wherein said cataloguing comprises referencing one or more cancer stem cell populations according to cancer/tumor type from which said one or more cancer stem populations were isolated.

9. The method of claim 6, wherein said cataloguing comprises referencing one or more cancer stem cell populations according to whether said one or more cancer stem populations express ganglioside.

10. The method of claim 6, wherein said cataloguing comprises referencing one or more cancer stem cell populations according to whether said one or more cancer stem populations express glycoconjugates observed in normal stem cells.

11. A dendritic cell activated against a cancer stem cell.

12. The dendritic cell of claim 11 activated against glioblastoma cancer stem cells.

13. The dendritic cell of claim 11, activated against bladder cancer stem cells.

14. The dendritic cell of claim 11, activated against breast cancer stem cells.

15. The dendritic cell of claim 11, activated against colon cancer stem cells.

16. The dendritic cell of claim 11, activated against.pancreatic cancer stem cells.

17. A cancer stem cell bank comprising:
multiple cancer stem cell populations produced by harvesting tumor tissue samples from a plurality of subjects and isolating cancer stem cells from individual tumor samples to produce a plurality of separate cancer stem cell populations; wherein said cancer stem cell populations are possessed by a single provider; and
a catalogue comprising information of predetermined characteristic information correlating with individual cancer stem cell populations.

18. The cancer stem cell bank of claim 17 further comprising:
multiple dendritic cell lines each being activated by using the isolated cancer stem cells, wherein said multiple dendritic cell lines are possessed by a single provider and
a catalogue comprising a reference to cancer/tumor type against which each dendritic cell line has been activated.

19. The activated dendritic cell bank of claim 18, wherein said bank comprises at least one dendritic cell line activated against glioblastoma cancer stem cells.

20. The dendritic cell of claim 11, wherein said cell is activated by subjection to a cancer stem cell, or antigen derived therefrom.

21. (canceled)

22. (canceled)

23. A method of treating a patient experiencing a cancer condition, said method comprising administering a therapeutically effective amount of a stem cell stimulating agent, and optionally, simultaneously, or sequentially, administering a chemotherapy agent and/or radiation treatment to the patient.

24. (canceled)
